(19) United States
(12) Patent Application Publication
(10) Pub. No.: US 2018/0340186 A1
(43) Pub. Date: Nov. 29, 2018

(54) CELL IMMORTALIZATION VIA VORTEX ELECTROPORATION GENE DELIVERY

(71) Applicant: President and Fellows of Harvard College, Cambridge, MA (US)

(72) Inventor: Soojung Claire Hur, Baltimore, MD (US)

(21) Appl. No.: 15/768,226

(22) PCT Filed: Oct. 14, 2016
(86) PCT No.: PCT/US2016/057117

§ 371 Date: Apr. 13, 2018

Related U.S. Application Data
(60) Provisional application No. 62/242,089, filed on Oct. 15, 2015.

(51) Int. Cl. C12N 15/85; C12N 5/07

(52) U.S. Cl. CPC C12N 15/85; C12N 5/06

(57) ABSTRACT

A method is provided to transform progenitor cells, fetal cells, stem cells or tumor cells, e.g., in a micro-fluidic device, with nucleic acid or protein.

### CROSS-REFERENCE TO RELATED APPLICATIONS

This application claims the benefit of the filing date of U.S. application Ser. No. 62/242,089, filed on Oct. 15, 2015, the disclosure of which is incorporated by reference herein.

### BACKGROUND

Circulating tumor cells (CTCs) from cancer patients' blood not only carry the information of events and/or mutations that the diseased cell recently underwent but also have presumably the highest metastatic potential. Thus, outcomes from various biological assays performed directly on freshly collected CTCs will expedite the identification of the best, up-to-date, patient-specific therapeutic strategies. However, those assays require dividing cells to examine efficacies of various therapeutic agents and their corresponding outcomes. To date, a robust mechanism to repeatedly passage adherent monolayers of CTCs has not been achieved because CTCs became senescent after a few passages (Yu et al., 2014).

### SUMMARY

The present disclosure provides methods to transform and optionally immortalize cells, e.g., “rare” or “atypical” cells, purified from a physiological sample of a patient, e.g., a physiological sample, e.g., a fluid sample such as urine, cerebrospinal fluid, saliva, ascities fluid, peritoneal fluid, pleural fluid, or blood, by delivering at least one vector, for instance, a plasmid, via vortex-assisted electroporation. In one embodiment, the cells are non-adherent cells. In one embodiment, the sample is a suspension of cells from a tissue sample, e.g., a tissue biopsy. The sample includes cells of different sizes (e.g., diameters) and “rare” or “atypical” cells of interest in the sample are, in one embodiment, larger cells. In one embodiment, the cells to be electroporated are “rare” cells in the population of cells in the sample, such as cells that represent less than 1%, 0.1%, 0.01% or less, of the population of cells in the physiological sample. In one embodiment, the cells to be electroporated are larger cells in the population of cells in the sample, such as cells with diameters that are 0.5%, 1%, 2%, 5% , 7%, 10%, 12% or greater than the diameter of a majority of differentiated cells in the population of cells in the physiological sample. In one embodiment, the cells are progenitor cells. In one embodiment, the cells are CTCs, endothelial progenitor cells, epithelial progenitor cells, or fetal nucleated red blood cells. In one embodiment, CTCs, or tumor stem cells, e.g., of epithelial (endoderm), mesenchymal (mesoderm) or endothelial (ectoderm) origin, are transformed with a vector that encodes a gene product, e.g., RNA or protein, or an isolated protein, that immortalizes the cells, for example, a vector to exogenously express Telomerase Reverse Transcriptase protein (TERT) in cells such as CTCs, thereby providing for their in vitro culture maintenance and growth. Successful immortalization allows ex vivo expansion of a subset of patient-derived cells of interest, which provides for clinical applications, e.g., personalized drug susceptibility testing, cancer genotyping as well as metastasis potential assessment, and regenerative medicine.

In one embodiment, the method employs a device that includes a microfluidic trap disposed along a microfluidic channel, the trap and channel having dimensions to create a fluid vortex within the trap to trap a particle of interest, e.g., cells (target cells) that represent less than 10%, 1%, 0.1%, 0.01% or less of the population of cells in a physiological sample. In one embodiment, the target cells have a diameter of about 12 to about 25 microns which is larger than differentiated cells such as leukocytes. In one embodiment, the device includes an electrode having interdigitated electrically isolated fingers positioned in the trap to create an electric field across the trap such that the electric field causes electroporation of a molecule, e.g., a nucleic acid vector, into cells.

In one embodiment, a device useful in the methods includes an array of microfluidic traps disposed along a set of microfluidic channels, the traps and channels having dimensions to create a fluid vortex within each trap to trap cells of interest. In one embodiment, the device includes an electrode structure having a set of interdigitated electrically isolated fingers positioned in each trap to create an electric field across the trap, and optionally a pair of pads to couple to a voltage source such that the electric field causes electroporation of molecules, e.g., a nucleic acid vector, in the fluid into cells.

In one embodiment, the method includes providing fluid containing particles of interest, e.g., a physiological fluid sample comprising cells of different sizes, to an array of traps positioned along multiple channels, the fluid provided at a pressure sufficient to cause vortex flow within the traps and trap one or more particles of interest, e.g., larger cells, in the traps and applying a voltage across an electrode structure. In one embodiment, the electrode structure has interdigitated electrodes formed in the traps to provide an electric field in the traps to cause electroporation of molecules in the fluid into the trapped particles.

In one embodiment, the method employs electrode array structure on an electrode substrate, the electrode array structure having an array of sets of interdigitated electrically isolated sets of finger electrodes and forming a channel and trap pattern in a device layer over the electrode substrate, such that each trap sealingly covers a corresponding set of interdigitated electrically isolated finger electrodes.

Also provided are methods of using cells immortalized by the methods described herein, e.g., in personalized or regenerative medicine. Immortalized patient-derived cell lines established by the described technology can be used to evaluate therapeutic index, to predict patients' tumor evolution in response to therapy, to identify new therapeutic agents, or to investigate new combination therapy regimens.

### BRIEF DESCRIPTION OF THE DRAWINGS

FIG. 1 is a block diagram of an electroporation chamber according to an example embodiment.

FIG. 2 is a block diagram of the electroporation chamber of FIG. 1 illustrating an interdigitated electrode structure according to an example embodiment.

FIG. 3 is a block schematic diagram of an electroporation device including an array of electroporation chambers and channels, with an electrode structure according to an example embodiment.

FIG. 4 is a block schematic diagram of cross connectors for an electrode structure according to an example embodiment.

FIG. 5 is a block schematic diagram of an array of electroporation chambers and corresponding electrode structure according to an example embodiment.

FIG. 6 is a block schematic diagram illustrating interdigitated finger electrodes within a chamber of an array of electroporation chambers according to an example embodiment.

FIG. 7 is a block schematic diagram of a SPICE model for an electroporation array according to an example embodiment.

FIG. 8 is a graph illustrating simulated voltage at multiple nodes of an electroporation array according to an example embodiment.

FIG. 9 is a schematic of an electroporator array, illustrating (a) an overview of the device, (b) photo of an assembled device, and (c) a microscopic image of 16 chambers. The scale bar represents 500 μm.

FIG. 10 is an exemplary method to isolate and electroporate cells for immortalization.

FIG. 11 shows exemplary uses for the immortalized and expanded atypical cells.

### DETAILED DESCRIPTION

In the following description, reference is made to the accompanying drawings that form a part hereof, and in which is shown by way of illustration specific embodiments which may be practiced. These embodiments are described in sufficient detail to enable those skilled in the art to practice the invention, and it is to be understood that other embodiments may be utilized and that structural, logical and electrical changes may be made without departing from the scope of the present invention. The following description of example embodiments is, therefore, not to be taken in a limited sense, and the scope of the present invention is defined by the appended claims.

An optimized electrode geometry for a microfluidic vortex based particle trapping array device facilitates efficient electroporation. In some embodiments, a 4-fold higher throughput (e.g., enhanced parallelization capability) and 10-fold lower operational voltage may be obtained over previous configurations. Some embodiments utilize micropatterned gold electrodes which may be seamlessly integrated with microfabrication process flows, allowing batch productions of the device.

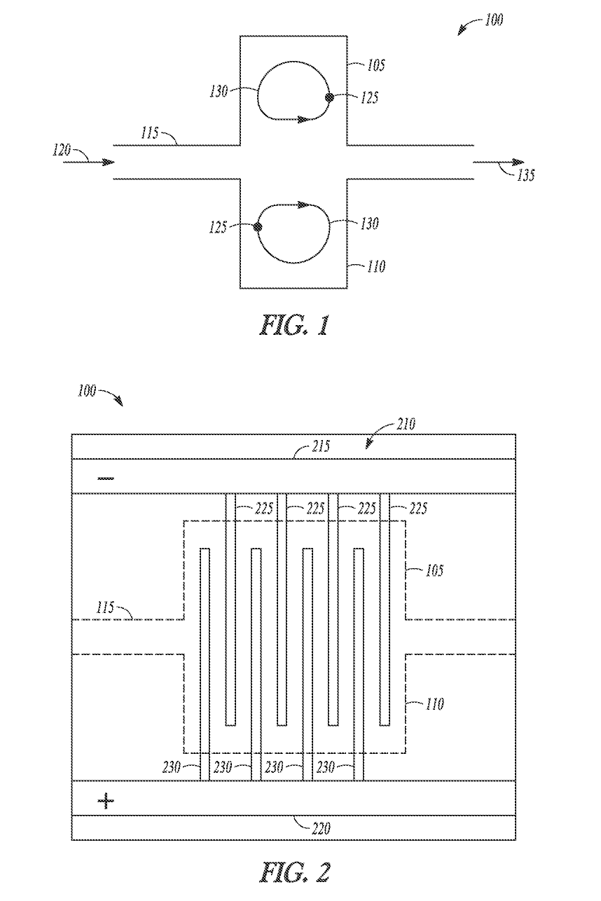

FIG. 1 is a simple block diagram of an electroporation device 100. A pair of electroporation traps 105 and 110 are bisected by a channel 115. Together, the traps 105 and 110, along with corresponding portion of the channel 115 form a chamber. Fluid through an input 120 of channel 120 contains particles 125, such as cells, which become trapped in a vortex flow 130 created in each trap 105, 110 when the fluid is flowing through the channel 115 with a Reynolds number of approximately 100. Fluid exits channel 115 via an output 135. Multiple such devices 110 may be assembled in an array consisting of multiple channels and multiple traps per channel. While opposing pairs of traps are shown, forming a chamber that is bisected by the channel, in further embodiments, traps may be formed on only one side of a channel, or alternate on different sides along the length of the channel. Various different geometries of traps may be used from rectangular to semi-circular or other arcuate or geometric shapes suitable for forming a vortex flow and capturing particles. The vortex, and orbit of the vortex may be modified by modifying the geometries and flow rates of the device.

FIG. 2 is a block diagram of the device 100 in FIG. 1 shown in further detail. The channel 115 and traps 105, 110 are shown as broken lines to facilitate showing a substrate layer having an electrode structure indicated generally at 210. The electrode structure 210 comprises a negative finger connective conductor 215 and a positive finger connective conductor 220 disposed on opposite sides of the traps 105 and 110 respectively. The negative finger connective conductor 215 comprises a plurality of electrode fingers 225 extending from the negative finger connective conductor 215 into the trap 105 and to near the end of trap 110. Similarly, the positive finger connective conductor 220 comprises a plurality of electrode fingers 230 extending from the positive finger connective conductor 220 into the trap 110 and to near the end of trap 105. By only extending to near the end of the respective traps, only one set of electrode fingers need be accounted for when creating a seal between the electrode substrate and a channel and trap layer that are coupled to form the device 100. The respective sets of electrode fingers are interleaved in an alternating manner to create the ability to generate an electric field when a voltage is applied across the connective conductors 215 and 220. Note that the positive and negative connotations of the electrode structure may be reversed in further embodiments.

In some embodiments, the electroporator device 100 is composed of two layers: A glass slide with patterned conductive electrodes (Au for example) on the surface enclosed with a fluidics layer having an array of cell trapping chambers and channels. The fluidics array may be formed of PDMS in one embodiment. Plastic (COP, COC, PMMA, PC) and other suitable materials may also be used. The slide with electrodes may be glass or other material suitable for forming conducting electrodes and mating with the fluidics layer. The electrode dimension, number of electrodes per chamber, and the gap distance between adjacent electrodes may be optimized such that the chamber area influenced by the electric field is maximized, a cell trajectory does not reside at the tip of the electrodes, thereby enhancing the uniformity of the electric field that cells are experiencing, and the electric field should be sufficient enough to electroporate cells without unwanted electrolysis and bubble formation.

In further embodiments, the electrical resistances at each location may be varied to have different electrical field profiles within each chamber. This is useful to help identify an electric field profile to electroporate a given cell type. The device can be tested with varied electrical resistance to identify the optimum condition. The device can then be changed to create a uniform electric field across all chambers to perform the electroporation. For both cases, the electric field in the chamber is uniform across the width or height of the chamber.

The ability to trap cells with a uniform size distribution positively contributes to having better molecular delivery efficiency and cell viability because the cell size distribution has significant implication for membrane permeabilization. When cells are exposed to an extracellular electric field, E, transmembrane potential, ΔVₘ=ƒE D cos θ, is induced in a cell. Here, ƒ is the weighting factor, which is the measure of how cells contribute on the applied electric field distribution, and D and θ are the cell diameter and the polar angle measured with respect to the external field, respectively. In order to transiently permeabilize cells without cell lysis, ΔVₘ should exceed the transmembrane potential, ΔVₛ, but remain below the irreversible electroporation threshold, ΔV_{c}. For mammalian cells, ΔVₛ is reported to range between 200 mV and 1 V, depending on pulse duration, while ΔV_{c} is expected to be greater than 1 V. Previous electroporation tests performed with cell lines that have a large size variation are reported to have low electroporation efficiency and viability since samples with higher heterogeneity in size are expected to have more number of cells having induced ΔVₘ that falls outside of the optimum range for successful transient permeabilization. Therefore, the current system's ability to pre-isolate cells with higher homogeneity in size may minimize undesirable consequences associated with a large cell size variation.

In a further embodiment, cross chamber voltage for each electroporation chamber may be modified individually by carefully tweaking the geometry of connecting electrodes and corresponding electric fields can be predicted using COMSOL and SPICE modeling. With a single injection of the given cell population, their responses to selected series of voltage magnitudes could be assessed simultaneously and rapidly. In addition, by setting up different outlets from the channels, cells, from the same batch but treated with different electroporation conditions or chemical doses, can be collected separately for parametric studies.

In still further embodiments, Au electrodes embedded in one or more chambers may serve as sensing electrodes as well as electric potential sources. With appropriate biochemical surface modification corresponding to target analytes, these electrodes, or a further set of electrodes in one or more chambers may be used to pick up minute electrical signal change (typically current) in correlation with the response of a trapped cell population to specific chemical/biological stimuli, e.g., nucleic acid vectors or isolated protein, applied in the chamber. The electrical component connected to the device may be modified accordingly to collect and amplify the signal while deducting background noise.

The use of an array may increase the overall throughput per batch and make the electroporation process more efficient and practical. However, simply adding more electroporation units does not guarantee replication of the same performance of the device with a single electroporation unit. The rationales for the optimum layout design of the electroporator array, especially the connecting electrode pattern include: (1) to maximize voltage efficiency of the electroporation array, (i.e., to minimize the voltage drop (waste) outside the cell-trapping chamber area); (2) to precisely predict electric fields being applied to orbiting cells and apply a uniform electric field across all chambers, or apply distinctive electric fields to different chambers to identify optimum conditions for given cells; and (3) to ensure sealing between PDMS and glass substrate. In some embodiments, wider connecting electrodes may be used to minimize the voltage variation across each chamber (trap) while a narrower electrode width is desired to ensure sealing.

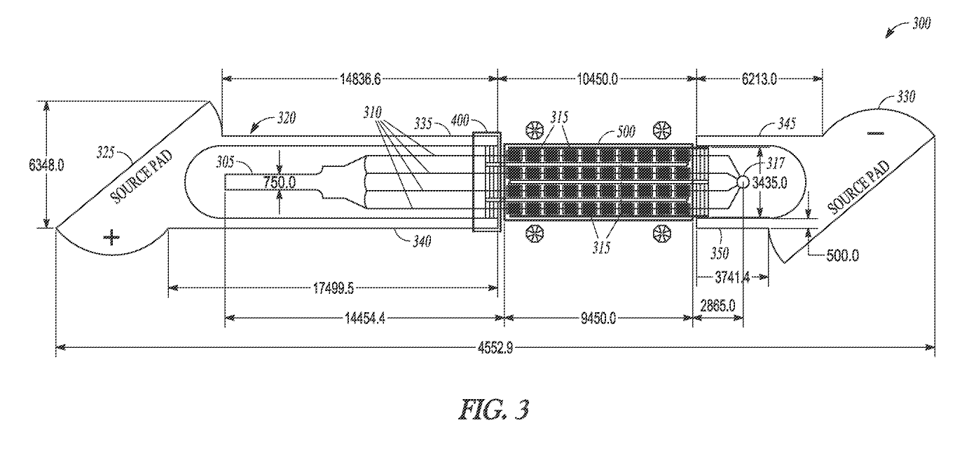

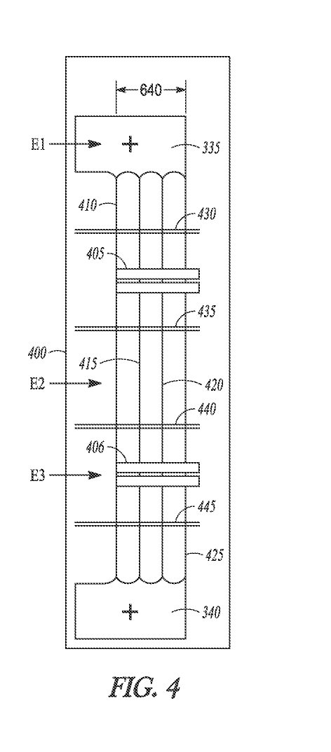

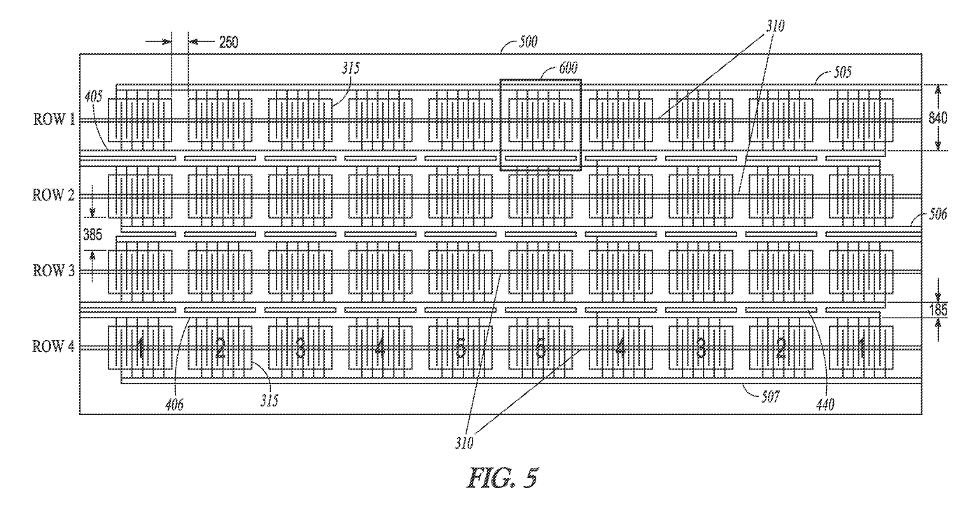

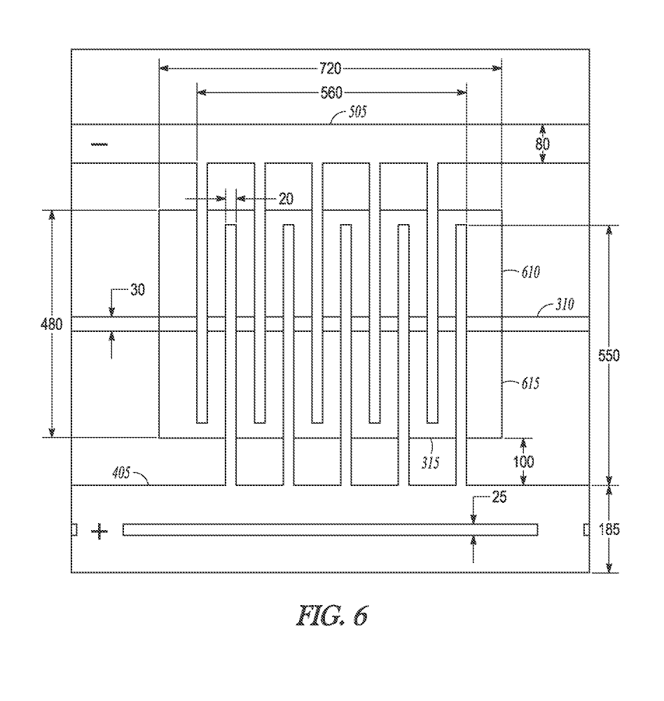

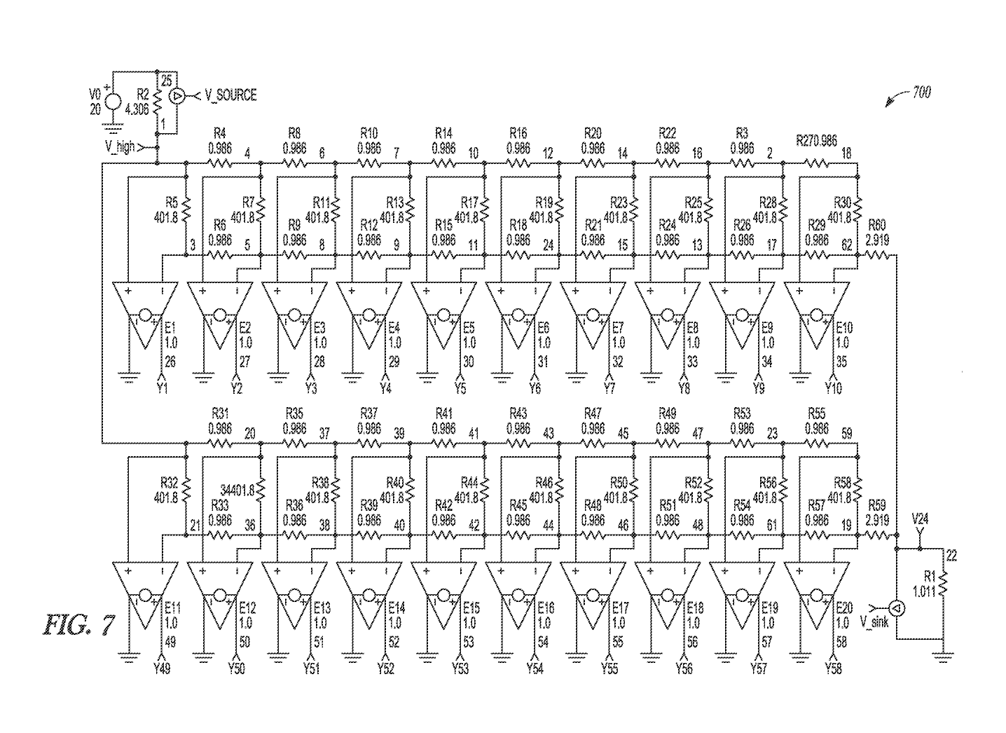

An example microscale electroporator symmetrical array is illustrated in FIG. 3 at 300, with FIGS. 4, 5, 6, and 9 showing further detail of elements of FIG. 3. In one embodiment, the array 300 includes an input fluid channel 305 that branches into four chamber channels 310 that each contains 10 chambers 315. The channels 310 proceed through the array of chambers and empty at 317. Each chamber comprises a pair of opposed traps extending from the chamber channels 310, as shown in further detail in FIG. 6. The array thus includes 40 chambers (4 rows×10 chambers per each row, i.e., a total of 80 cell trapping vortices per device at its full capacity). In one embodiment, sets of four neighboring chambers (2×2) are positioned such that they can be fit into a single field of view of a camera (not shown) when a 4× objective is used. 10 multi-point automated imaging sequences may be used to visualize and monitor all 80 cell-trapping vortices in real time during electroporation. In further embodiments, other sensing mechanisms may also be employed.

An electrode structure is illustrated generally at 320 and is formed of various widths of conductors, which may also be referred to as conductors or connecting wires. A source pad 325 provides a convenient conductive pad for coupling to a positive voltage source, and a source pad 330 provides for coupling to a negative voltage source. The sources may be reversed in one embodiment, and may be variable voltage or DC voltage sources. Variable voltages include square wave sources and AC sources to name a few. The electrode structure 320 also includes two connective conductors indicated at 335 and 340 to couple to further finger connective conductors shown in further detail with interdigitated finger conductors in FIG. 5. Similarly, the negative source pad 330 branches into two connective conductors indicated at 345 and 350, which further branch into finger connective conductors and interdigitated finger conductors, forming the array of finger conductors to create a desired electric field in the traps for electroporation.

Sets of cross conductors is illustrated at 400, and serve to couple the finger connective conductors, indicated at 405 and 406 to each other, providing a failsafe for broken conductors and further enhancing the voltage uniformity of the electrodes in each trap. As seen in FIG. 4 at 400, there are four cross conductors 410, 415, 420, and 425 and addition orthogonal cross conductors 430, 435, 440, and 445 coupled to the connective conductors 335 and 340. Similar cross conductors may be used for connective conductors 345 and 350 as shown in FIG. 3.

The two finger connective conductors 405 and 406 are sufficient to couple to positive interdigitated finger conductors in each of the chambers. Conductor 405 extends between the first two rows of chambers, and conductor 406 extends between the last two rows of chambers. The finger connective conductors coupled to the negative source pad 330 branch into three, extending along the outsides of the first and fourth rows of chambers and between the second and third rows of chambers. In one embodiment, the finger connective conductors extending between rows may comprise a conductor for each row, which may be periodically interconnected along their length.

Within each PDMS cell trapping chamber (Lc=720 μm; Wc=480 μm; Hc=70 μm), there are 5 pairs of interdigitated Au electrodes (20 μm×450 μm) in one embodiment. For each row, electrodes with the same polarity are connected with a single wire (denoted as E3 corresponding to 405 and 406, WE3=80 um), transferring electric signals from the source. Several connecting points may be formed between two adjacent wires to eliminate chances of device malfunctions due to fabrication defects. The connecting wires were designed to have two sections of varied electrode widths. The first section (denoted as E1 corresponding to 335 and 340), which is immediately after the electric source pad 325, are designed to have electrode length and width of LE1≈16 mm and WE1=500 μm, respectively. The first section electrodes are then branched into the second sets of 4 connecting electrodes 410, 415, 420, 425, denoted as E2, whose length and width are LE2≈3 mm and WE2=20 μm. The first and second sections of electrodes are located orthogonally in order to deliver electric signals to each row of the electroporation chambers. The purpose of four repeating E2 cross connectors may be to lower the overall electrical resistance across those electrodes (i.e., lowering the voltage loss) by parallelizing resistors while eliminating the leakage of injected fluids at the site where Au electrodes are in contact with PDMS. The number and widths of the repeating connectors can be varied in order to intentionally vary electric fields for each chamber. The width of the electrodes, directly under the edge of electroporation chamber (where PDMS and glass substrate are bonded) should be smaller than 20 um for a 300 nm thick Au layer. Otherwise, the injected fluid may leak between the chamber and substrate where the connectors traverse the substrate under the edge of the chamber. In one embodiment, the value of WE2=20 μm is small enough to help ensure sealing, such as irreversible sealing, between a substrate layer on which the electrodes are formed and a device layer in which the traps and channels are formed. In one embodiment, the electrodes may be formed on a glass slide with micropatterned 300 nm-thick Au electrodes and the traps and channels may be formed in a PDMS layer. These values are just examples, and may be varied in further embodiments.

FIG. 5 provides a larger view of the array at 500. Note that the reference numbers used in FIGS. 3, 4, 5, and 6 are consistent. The finger connective conductors 405 and 406 are illustrated running between the rows from positive source pad 325. Further finger connective conductors 505, 506, and 507 are shown running outside of the outer rows (505 and 507) and between (506) the second and third rows from the negative source pad 330. A chamber area identified by a box 600 is shown in further detail in FIG. 6.

Chamber area 600 shows the chamber 315 and channel 310 bisecting a pair of traps 610 and 615. Positive finger connective conductor 405 is shown at one side of chamber 315, with negative finger connective conductor 505 shown at an opposite side of chamber 315. Note that the finger connective conductors are shown running in substantially the same direction as the channels, and are located outside the boundaries of the chamber.

Positive finger electrodes 620 run from the positive finger connective conductor 505 toward the negative finger connective conductor 605, and negative finger electrodes 625 run from the negative finger connective conductor 605 to the positive finger connective conductor 505. Both sets of finger connective conductors in one embodiment interdigitate and extend to almost a far end of the chamber, without going further than the chamber wall to ensure a better seal of the chamber to the electrode substrate. In one embodiment, there are five each of the positive finger conductors and negative finger conductors interleaved to form a uniform electric field distribution over the area of the chamber corresponding to the electrode substrate. The field strength weakens as the distance in the chamber orthogonal to the electrodes increases.

Square waves may show slightly better performance compared to that of sine wave in terms of electroporation efficiency when the identical peak AC voltage is applied. The effectiveness may be related to the root mean square (i.e., Vrms) of the voltage, rather than the absolute magnitude, Vₚₖ, for electroporation.

Due to the minute gap between the two adjacent electrodes (40 μm), the correct frequency choice should be made for the proper functioning of the electroporator array. Note that adjacent electrodes of opposite polarity are electrically isolated from each other except for any conductivity provided by the fluid.

If the frequency is too low (f<10 kHz, the lower limit of the feasible frequency range for the current configuration), bubbles may be generated inside the electroporation chambers. Bubble generation may interfere with cell trapping stability and may also damage the Au electrodes. On the other hand, if the frequency is too high, such as f>40 kHz in one embodiment, the rapid shift of electric field polarization may diminish the electroporation process. In one embodiment, 20 kHz was chosen as the optimal frequency for the current electroporator array with cells of interest.

Various pulse numbers were tested to evaluate whether extra pulses would lead to dramatic performance enhancement. Both the electroporation efficiency and cell viability were similar when either 6 or 10 pulses were applied, especially at higher voltage (i.e., >12 Vₚₖ), suggesting that within the tested voltage range, using 10 pulses will not cause adverse effects to some cells.

Based on the discussion above, the standard parameters used for some experiments to perform electroportation were set to be a square wave with 20 kHz, 10 pulses, 1 ms pulse width and 1 s interval between each pulses unless otherwise specified. Adopting the standard parameter, electroporation at different voltage levels were conducted and cells were collected downstream into 96 well plates after electroporation for evaluation. Further analysis revealed Vₚₖ=14.5 V as an optimal voltage for HEK 293. It was also noticed that the total cell number collected in the well plate dropped dramatically at relatively higher voltage ranges (>17 Vₚₖ). Presumably, it is due to either the interference of bubble generated inside the chamber or trapped cell bursting occurred at high voltage. Therefore, when 17<Vₚₖ, the electroporator array suffers from low number of collected cells although the efficiency and viability percentage are still comparable to that of the optimal voltage.

In further embodiments, the voltage may be selected to intentionally lyse or burst selective cells. Different pulse parameters may also be selected. Such a process may be used to create more pure subpopulations of cells to collect downstream.

Thus, after target cells are trapped in vortices, various voltages can be applied to trapped cells. Voltages higher than the transmembrane potential of cells may cause irreversible cellular membrane damage to achieve “controlled” cell lysis. Here, the applied voltage can be lower than the voltage inducing electrolysis (i.e., to avoid severe bubble issue). Gradual increase in voltages would enable sequential cell lysis. The intracellular components from burst cells may also be sequentially sampled downstream at each cell-lysis event for further concentration and analysis.

One of the parameters for the performance evaluation of electroporation is the apparent electric field intensity that cells are exposed to. The conventional approach for electric field estimations is to divide the applied voltage magnitude by the distance of two vertical electrodes between which cell solutions reside. However, with the use of planar electrodes located at the bottom of the chamber, the electric field distribution may vary widely across the cell solution over the depth of the chamber. In addition to consequences caused by the planar electrode configurations, the fact that cells are moving in suspension during electroporations suggests that the simple average estimation may not be accurate or reliable. Simulations using COMSOL provide a better understanding of the electric field distribution inside the chamber, particularly at different heights of the chamber and along the cell trajectory. Based on some simulation results, the resistance of single chamber filled with DPBS, whose electrical conductivity is 1.4 S/m, during electroporation was estimated at about 430 Ohm.

During electroporation, both the Au patterns and the conducting solution injected in the microchannel function together as a complex circuit composed of various electrical resistors. The applied voltage reading from the power supply does not necessarily represent the actual voltage across each chamber due to the complexity of the device layout. The use of SPICE simulations prior to electrode manufacturing may be used for verification of circuit/device operation at the transistor level, and may provide guidance for design optimization of devices having varying array and channel sizes and layout.

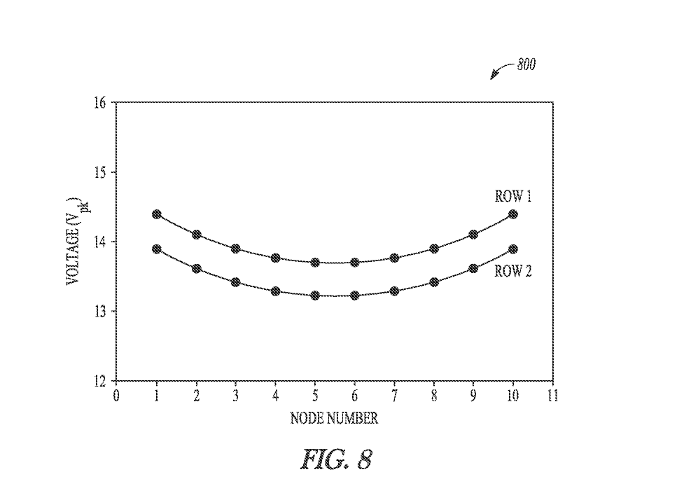

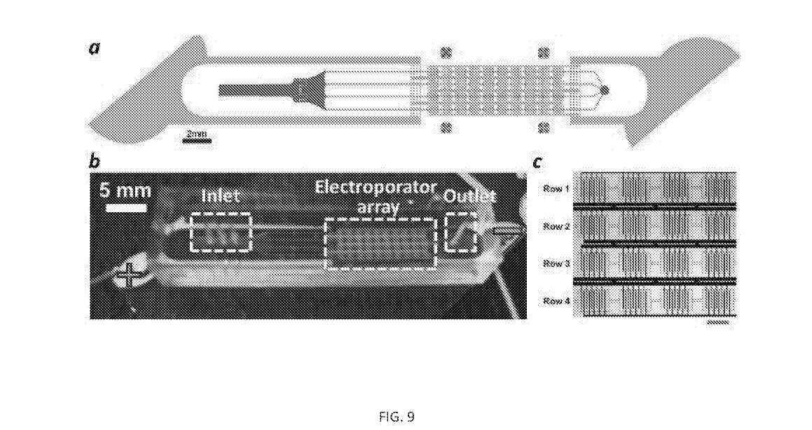

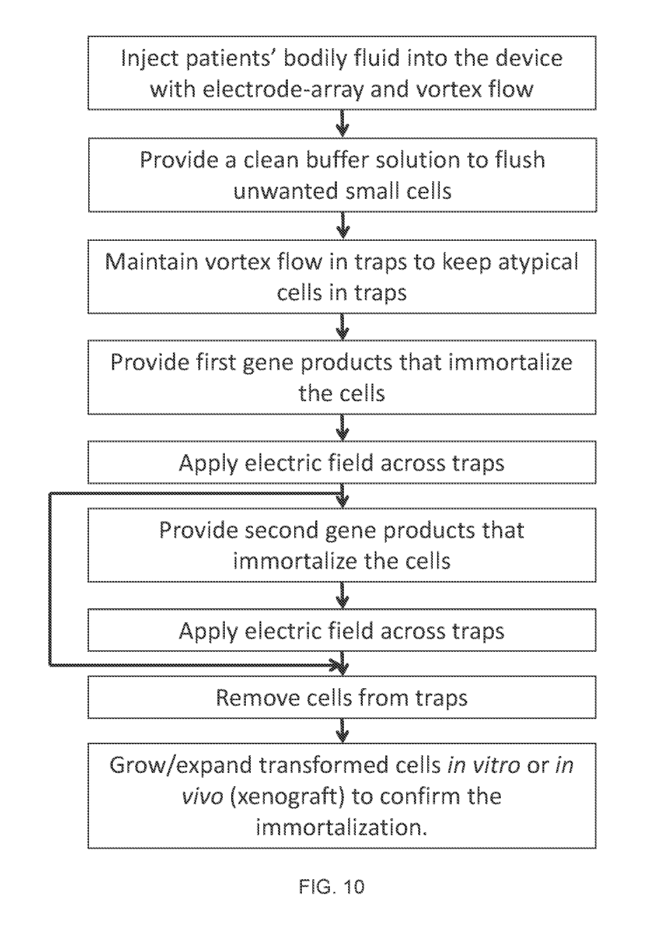

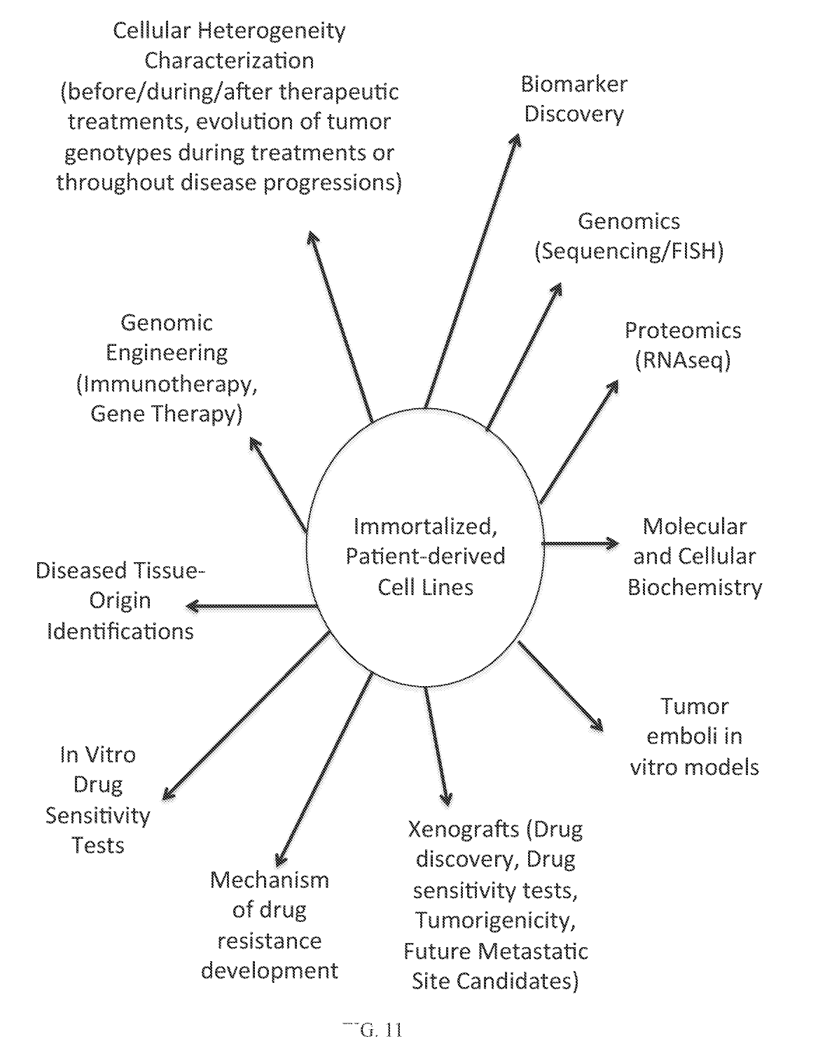

FIG. 7 illustrates a SPICE model 700 of an upper half of the array 500. The lower half of the array shares exactly the same structure. Varying the widths of the different connecting wires results in different resistances, which may be taken into account by the SPICE model. Using the SPICE model 700, cross chamber voltage variation of the first two rows of the array with an input peak voltage, Vₚₖ, of 20 volts is illustrated in a graph 800 in FIG. 8 showing the voltage at each node. Each circle or node on the graph corresponds to a chamber. The same model may be used to design an electrode array with intentionally varied resistances at each chamber in further embodiments. Such embodiments may be useful in identifying and confirming electrode arrays to obtain desired performance in different situations, such as for electroporating, lysing, or electrochemical-sensing various particles or cells.

Criteria which may be used to assess the electroporator array performance include: (1) chamber voltage efficiency, which refers to how much fraction of applied voltage actually available at the cell trapping chambers for sufficient electroporation (i.e., part of the applied voltage would be inevitably lost via the connecting Au patterns; (2) Cross chamber voltage variation, which represents that, within the chamber array, how variable the voltages are among individual chambers. Using row 4 in FIG. 5 as an example, chambers 1 to 5 have different cross chamber voltages due to the variation in lengths of the finger connective conductors. Typically the more chambers serially connected in one row and/or the more rows one device consists of in parallel, the larger the chamber-to-chamber voltage variation tends to get. For optimization, it is desired to optimize electroporation effectiveness with minimal voltage, and to have uniform electroporation effectiveness across all the chambers on the same device. Based on the calculation using a SPICE model, the balance between the two parameters may be achieved with 80% voltage efficiency and a chamber voltage variation less than 8% for 40 chambers (trap pairs) per device using the dimensions shown. The particular dimensions utilized for the electrode structure and channel and trap sizes may be varied significantly in further embodiments. More or fewer channels and traps per channel may be used. The electrode structure and layout may also be modified. The dimensions, such as length (straight vs. serpentine), width, and number of parallel arrays of electrodes can be modified to create desired electric field profiles within the electroporation chambers. Serpentine geometry may be useful for applications benefiting from higher electrical resistances (Joule heating or large voltage drop across electrodes). The materials used for the electrodes may also be varied, with Au being one material that may be incorporated with other materials, or other conductive materials or combinations of materials may be used, such as platinum, copper, and aluminum for example.

Due to the “independent”, yet “flexible”, nature of the device having a wide range of modifiable parameters (e.g., chamber geometry, chamber height, fluid conductivity, electrode geometry and electric field distribution), the device may serve as a fine model to combine with theoretical studies such as electrolysis and electroporation mechanism to provide experimental observation as well as validation with an aid of visualization equipment (e.g., high speed camera) and imaging analysis tools (e.g., computer vision algorithms).

In still further embodiments, the electrode array may be used to conduct fundamental studies on electrolysis and determine the thickness of electrical double layer of given conductive solution. This can be very useful tool for designing better next-generation electrical biosensors, and electroporator and/or cell lysis devices.

In one embodiment, the microscale electroporator array includes 40 chambers with the geometry adopted from the Vortex chip proven to have superior rare cell purification capability (FIG. 9). Each cell-trapping chamber contains 5 pairs of interdigitated Au electrodes (Wₑ=20 μm). Extensive COMSOL and SPICE simulations were conducted prior to electrode fabrications to predict the electrode design yielding an optimum performance. The electroporator array was fabricated using well-developed microfabrication and soft lithography processes and microfluidic cell trapping chamber arrays were enclosed with glass slides with patterned Au electrodes. Cells trapped in microscale vortices were visualized in real-time to monitor the electroporation process and processed cells were collected off-chip for downstream analysis. A membrane impermeable fluorescent molecule was delivered into HEK293 cells to optimize electroporation parameters. Square waveforms with varied frequencies (10˜40 kHz) and peak-voltages (10˜20Vₚₖ) were utilized to perform electroporation.

In one embodiment, electroporation conditions for tested cell lines are about 10 pulses with square waveform at about 20 kHz frequency, 14Vₚₖ, 10 ms pulse width and is pulse interval. 20 kHz was chosen as the optimal frequency because lower frequency (about 10 kHz) induced unwanted electrolysis while higher frequency (e.g., 40 kHz) resulted in unacceptable electroporation efficiency. Operational voltage typically ranges from 10 to 20Vₚₖ for the tested cell lines, but the optimal voltage, yielding efficient electroporation with high viability, would vary depending on cell types (e.g., the optimum Vₚₖ were 14 and 15 for HEK293 and MCF7, respectively). In one embodiment, the conditions are voltage of about 5˜20 Vrms, Square or Sine wave or DC pulses, 5 to 30 pulses, and a frequency of about 15˜40 kHz.

When macromolecules conjugated with fluorophores were introduced into electroporated HEK293 or MCF7 cells. The fluorescent intensities of cells were proportionally increased with increasing incubation times for both protein and siRNA delivery. p53-wildtype MCF7 cells were transfected with miR-29, which has been proved to activate p53 protein pathway in cancerous cells, inducing apoptosis. Electroporation itself did not affect viability nor alter morphology of processed cells compared to that of control. However, cells transfected with miR-29 exhibited apoptotic phenotype with greater amount of externalized phosphatidylserine, which was visualized by Annexin V staining. Furthermore, cells transfected with miR-29 via electroporation exhibited higher apoptotic tendency although miRNA amount was 50% lower than transfection via lipofectamine.

The system has great potential to be a versatile multi-molecule delivery system, enabling high-throughput, cost-effective and rare cell transfections.

The present disclosure allows for purification of rare numbers of cells, e.g., CTCs, stem cells or progenitor cells, from physiological samples such as blood, and direct injection of plasmids of interest while the cells are in the microfluidic device. The cell trapping chamber geometry provides for enhanced CTC-purification efficiency without sacrificing electroporation efficiency or uniformity. For example, combinations of hTERT plasmid with various cell-type specific immortalization promoters, including but not limited to c-myc, v-myc, ZNF217 and RhoA, are delivered into living CTCs promptly after purification via electroporation to create immortalized patient-specific tumor origin cells with genetic preservation.

Successful intracellular delivery of multiple molecules to cells via electroporation provides for immortalized cell lines, which can be cultured and propagated in vitro. Since electroporated cells are collected off-chip after the procedure in dispersed single-cell form, single-cell derived colony picking for downstream analysis is provided. Individual colonies are presumably isogenic, enabling identification in heterogeneity among collected atypical cells (e.g., tumor-initiating/cancer stem cells). The established cell lines can be employed in patient-specific assays, including but not limited to biomarker discovery, genomics, proteomics, drug sensitivity tests, drug combination assays, drug resistance mechanism studies, drug discovery, elucidating genetic and molecular basis of the current disease states (e.g., sites of mutation, metastatic potential, diseased tissue-origins). Established cell lines continuously provide cells whose quantity is sufficient enough to conduct conventional flow cytometry studies, required for subpopulation classifications with statistical significance. Classified/sorted cells can be used for further downstream analyses (e.g., heterogeneity profiling, xenografts, etc.). In particular, for CTCs, in vitro drug efficacy/sensitivity tests that are conducted directly on immortalized patient-derived CTC cell lines can provide guidance to modify, diversify and improve personalized therapeutic strategies. Moreover, the cells also can be transplanted in mice for human tumor xenografts to investigate mechanisms, contributing to metastasis, invasion and/or responses and resistances to therapeutic agents.

Furthermore, immortalized cell lines can be further genetically modified (e.g., deletion or insertion of gene of interests), to evaluate gene therapy or immunotherapy potentials by incorporating genomic engineering methods, such as Transcription Activator-Like Effector Nucleases (TALENs), Zinc-finger Nucleases (ZFNs) and Clustered Regulatory Interspaced Short

Palindromic Repeats (CRISPR-cas9). Immortalized cells can be further modified to express luciferase to evaluate tumor growth through in vivo bioluminescent imaging in CTC derived xenograft model.

The invention will be described by the following non-limiting examples.

### EXAMPLES

The delivery of various macromolecules into cells is a crucial but challenging task for biological and clinical applications, but existing technologies struggle to simultaneously achieve high transfection efficiency and low cytotoxicity. The most common immortalization processes rely on use of the delivery of infection viral vectors. Putting potential risks associated with viral genome integration aside, those procedures are either capable of transduction of actively dividing cells (retroviral vector) or having limited insert sizes (lentiviral vector). Moreover, conventional physical gene delivery mechanisms, including electroporation, are inapplicable for CTCs because high cytotoxicity accompanied by those mechanisms cannot be translated to an infinitesimal number of fragile CTCs (<100 cells per 10 mL of blood).

To address these shortcomings, a microfluidic electroporator array was developed that is parallelizable and capable of delivering a wide range of molecules commonly used in biology. The system can successfully deliver protein, siRNA and miRNA into various cell types at 10-fold lower operational voltages with 4-fold higher throughput compared to that of the previous configurations (see, e.g., U.S. Pat. No. 9,039,109 B2).

The present disclosure allows for purification of rare numbers of cells, e.g., CTCs, stem cells or progenitor cells, from physiological samples such as blood and direct injection of plasmids of interest while those cells are still in the microfluidic device. The cell trapping chamber geometry in the updated configuration provides for enhanced CTC-purification efficiency without sacrificing electroporation efficiency or uniformity. For example, combinations of hTERT plasmid with various cell-type specific immortalization promoters, including but not limited to c-myc, v-myc, ZNF217 and RhoA, are delivered into living CTCs promptly after purification via electroporation to create immortalized patient-specific tumor origin cells with genetic preservation.

Thus, in Example 1, the a method is provided that employs a device comprising:

a microfluidic trap disposed along a microfluidic channel, the trap and channel having dimensions to create a fluid vortex within the trap to trap a particle of interest such as rare cells in physiological fluid; and
an electrode having interdigitated electrically isolated fingers positioned in the trap to create an electric field across the trap such that the electric field causes electroporation of a molecule into the particle of interest.

2. The method of example 1 wherein the electrode is formed on a plane of a first substrate and is enclosed with a first layer having the channel and trap formed therein.

3. The method of example 2 wherein the interdigitated electrically isolated fingers are interdigitated within the trap.

4. The method of any of examples 2-3 wherein the trap comprises a pair of traps opposed from the channel and wherein the electrode comprises:

a first finger connective conductor running in the same direction as the channel and disposed outside one side of the pair of traps;
a second finger connective conductor running in the same direction as the channel and disposed outside the other side of the pair of traps; and
wherein the fingers run from each finger connective conductor toward the other finger connective conductor to form an interdigitated array of electrodes.

5. The method of any of examples 2-4 wherein the first substrate comprises glass, the electrodes comprise gold, and the first layer comprises PDMS.

6. The method of any of examples 1-5 wherein the device comprises an array of channels, each having a plurality of traps and electrodes.

7. The method of any of examples 5-6 wherein the electrodes include conductive pads and connective conductors coupled to the interdigitated fingers, the conductive pads to couple to plus and minus terminals of a voltage source.

8. The method of example 7 wherein the electrodes are patterned to minimize voltage variations between traps.

9. The method of example 8 wherein the electrode pads are wider than the connective conductors, and wherein the electrodes further comprise: multiple cross conductors to couple the connective conductors together; and a plurality of finger connective conductors coupled between the connective conductors and sets of the interdigitated fingers, wherein the connective conductors are wider than the finger connective conductors to minimize voltage variation across chambers.

10. The method of example 9 wherein the electrode pads, connective conductors, and finger connective conductors are disposed outside each trap.

11. The method of any of examples 9-10 wherein the trap comprises a pair of traps opposed from the channel and wherein:

each channel has a first finger connective conductor running in the same direction as the channel and disposed outside one side of the pair of traps;
each channel has a second finger connective conductor running in the same direction as the channel and disposed outside the other side of the pair of traps; and
wherein the fingers run from each finger connective conductor toward the other finger connective conductor to form an interdigitated array of electrode fingers in each pair of traps.

12. The method of any of examples 6-11 wherein each channel comprises at least 20 traps.

13. The method of any of examples 1-12 wherein the electrode comprises patterned gold.

Also provided is example 14 is a method that employs a device comprising:

an array of microfluidic traps disposed along a set of microfluidic channels, the traps and channels having dimensions to create a fluid vortex within each trap to trap a particle of interest such as rare cell types in physiological fluid; and
an electrode structure having a set of interdigitated electrically isolated fingers positioned in each trap to create an electric field across the trap, and a pair of pads to couple to a voltage source such that the electric field causes electroporation of molecules in the fluid into the particles of interest.

15. The method of example 14 and further comprising an input coupled to the set of microfluidic channels for providing fluids containing the particles of interest and selected molecules, and an output to remove fluid from the channels.

16. The method of any of examples any of examples 14-15 wherein the electrodes include finger connective conductors coupled to the interdigitated fingers, wherein the electrode pads are wider than the connective conductors, and wherein the electrodes further comprise:

multiple cross conductors to couple the connective conductors together; and
a plurality of finger connective conductors coupled between the connective conductors and sets of the interdigitated fingers, wherein the connective conductors are wider than the finger connective conductors to minimize voltage variation across chambers.

Further provided is example 17, which provides a method comprising:

providing physiological fluid containing cells of interest to an array of traps positioned along multiple channels, the fluid provided at a pressure sufficient to cause vortex flow within the traps and trap one or more rare cells in the traps; and
applying a voltage across an electrode structure, the electrode structure having interdigitated electrodes formed in the traps to provide an electric field in the traps to cause electroporation of molecules in the fluid into the cells.

18. The method of example 17 wherein the electrode structure is formed with different connection widths.

19. The method of any of examples 17-18 wherein electrode dimensions are selected to promote variations in electric field profiles in different chambers to identify electrode dimensions that produce a desired electric field profile.

20. The method of example 19 wherein the desired electric field profile is suitable for optimum electroporation or cell lysis.

Further provided in example 21 is a method comprising:

forming an electrode array structure on an electrode substrate, the electrode array structure having an array of sets of interdigitated electrically isolated sets of finger electrodes; and
forming a channel and trap pattern in a device layer over the electrode substrate, such that each trap sealingly covers a corresponding set of interdigitated electrically isolated finger electrodes.

22. The method of example 21 wherein the electrode array structure comprises finger connective conductors running in the same direction as the channels and positioned outside an area enclosed by the traps.

In Example 23, a method to electroporate circulating cells is provided comprising: introducing a physiological fluid sample comprising cells of different sizes that include progenitor cells, fetal cells, stem cells or tumor cells, to a device having a microfluidic trap disposed along a microfluidic channel, the trap and channel having dimensions to create a fluid vortex within the trap to trap cells in the sample that are larger in diameter than the diameter of a majority of cells in the sample and an electrode having interdigitated electrically isolated fingers positioned in the trap to create an electric field across the trap; maintaining a fluid vortex in the trap to isolate the larger cells that include progenitor cells, fetal cells, stem cells or tumor cells from smaller cells; introducing a nucleic acid vector encoding a gene product or an isolated protein to the trap having the isolated larger cells; and applying the electric field to cause electroporation of the nucleic acid vector or the isolated protein into the isolated larger cells.

24. The method of example 23 wherein the electrode is formed on a plane of a first substrate and is enclosed with a first layer having the channel and trap formed therein.

25. The method of example 24 wherein the interdigitated electrically isolated fingers are interdigitated within the trap.

26. The method of example 24 wherein the trap comprises a pair of traps opposed from the channel and wherein the electrode comprises: a first finger connective conductor running in the same direction as the channel and disposed outside one side of the pair of traps; a second finger connective conductor running in the same direction as the channel and disposed outside the other side of the pair of traps; and wherein the fingers run from each finger connective conductor toward the other finger connective conductor to form an interdigitated array of electrodes.

27. The method of example 24 wherein the first substrate comprises glass, the electrodes comprise gold, and the first layer comprises PDMS.

28. The method of example 23 wherein the device comprises an array of channels, each having a plurality of traps and electrodes.

29. The method of example 27 wherein the electrodes include conductive pads and connective conductors coupled to the interdigitated fingers, the conductive pads to couple to plus and minus terminals of a voltage source.

30. The method of example 27 wherein the electrodes are patterned to minimize voltage variations between traps.

31. The method of example 28 wherein the electrode pads are wider than the connective conductors, and wherein the electrodes further comprise:

multiple cross conductors to couple the connective conductors together; and
a plurality of finger connective conductors coupled between the connective conductors and sets of the interdigitated fingers, wherein the connective conductors are wider than the finger connective conductors to minimize voltage variation across chambers.

32. The method of example 29 wherein the electrode pads, connective conductors, and finger connective conductors are disposed outside each trap.

33. The method of example 29 wherein the trap comprises a pair of traps opposed from the channel and wherein:

each channel has a first finger connective conductor running in the same direction as the channel and disposed outside one side of the pair of traps; each channel has a second finger connective conductor running in the same direction as the channel and disposed outside the other side of the pair of traps; and
wherein the fingers run from each finger connective conductor toward the other finger connective conductor to form an interdigitated array of electrode fingers in each pair of traps.

34. The method of example 23 wherein each channel comprises at least 20 traps.

35. The method of example 29 wherein the electrode comprises patterned gold.

36. The method of any one of examples 23 to 35wherein the physiological sample comprises blood, saliva, ascities fluid, cerebrospinal fluid or urine.

37. The method of any one of examples 23 to 36 wherein the gene product or protein comprises TERT.

38. The method of any one of examples 23to 37 wherein the vector is a plasmid.

39. The method of any one of examples 23 to 38 wherein the larger cells are circulating tumor cells, fetal cells, epithelial progenitor cells or endothelial progenitor cells.

In Example 40, method to electroporate circulating cells is provided comprising: introducing a fluid having a physiological sample comprising cells of different sizes that include progenitor cells, fetal cells, stem cells or tumor cells, to a device comprising an array of microfluidic traps disposed along a set of microfluidic channels, the traps and channels having dimensions to create a fluid vortex within each trap to trap cells in the sample that are larger in diameter than the diameter of a majority of cells in the sample and an electrode structure having a set of interdigitated electrically isolated fingers positioned in each trap to create an electric field across the trap, and a pair of pads to coupled to a voltage source; maintaining a fluid vortex in the trap that results in isolation of the larger cells that include progenitor cells, fetal cells, stem cells or tumor cells from smaller cells; introducing a fluid having a nucleic acid vector encoding a gene product or an isolated protein to the traps; and applying the electric field to cause electroporation of the nucleic acid vector or isolated protein into the isolated larger cells.

41. The method of example 40 and further comprising an input coupled to the set of microfluidic channels for providing fluids containing the cells and the vector or isolated protein molecules, and an output to remove fluid from the channels.

42. The method of example 40 wherein the electrodes include finger connective conductors coupled to the interdigitated fingers, wherein the electrode pads are wider than the connective conductors, and wherein the electrodes further comprise: multiple cross conductors to couple the connective conductors together; and a plurality of finger connective conductors coupled between the connective conductors and sets of the interdigitated fingers, wherein the connective conductors are wider than the finger connective conductors to minimize voltage variation across chambers.

43. The method of any one of examples 40 to 42 wherein the physiological sample comprises blood, saliva, ascities fluid, cerebrospinal fluid or urine.

44. The method of any one of examples 40 to 43 wherein the gene product or protein comprises TERT.

45. The method of any one of examples 40 to 44 wherein the vector is a plasmid.

46. The method of any one of examples 40 to 45 wherein the larger cells are circulating tumor cells, fetal cells, epithelial progenitor cells or endothelial progenitor cells.

Electroporated patient-derived atypical cells can be collected off-chip and expanded in vitro to create immortalized patient-specific cell lines. The established cell lines can be used to determine better therapeutic agents via drug sensitivity assays, xenografts and/or genomic editing tools. For instance, the cell lines established by immortalizing patient-derived atypical cells can be cultured and propagated in vitro, providing sources of cells useful, for example, to identify better therapeutic strategies that individual patients respond most effectively via patient-specific drug sensitivity assays, patient-derived xenograft tumor model constructions, and/or genomic editing technologies.

Although a few embodiments have been described in detail above, other modifications are possible. For example, the logic flows depicted in the figures do not require the particular order shown, or sequential order, to achieve desirable results. Other steps may be provided, or steps may be eliminated, from the described flows, and other components may be added to, or removed from, the described systems. Other embodiments may be within the scope of the following claims.

The following statements are potential claims that may be converted to claims in a future application. No modification of the following statements should be allowed to affect the interpretation of claims which may be drafted when this provisional application is converted into a regular utility application.

### REFERENCES

### Castanotto and Rossi, Nature, 457: 426 (2009).

Garzon et al., Ann. Rev. Med., 60: 167 (2009).
Lipps et al., Biol. Chem., 394: 1637 (2013).

### Maheswaran & Haber, Cancer Res., 75: 2411 (2015).

Park et al., Nature Struct. & Mol. Biol., 16: 23 (2009).

### Plaks et al., Science, 341: 1186 (2013).

### Sollier et al., Lab on a Chip, 14: 63 (2014).

Vickers et al., Anal. Chem., 86: 10099 (2014).

### Yu et al., Science, 345: 216 (2014).

Yun and Hur, Lab. Chip, 13: 2764 (2013).

All publications, patents and patent applications are incorporated herein by reference. While in the foregoing specification, this invention has been described in relation to certain preferred embodiments thereof, and many details have been set forth for purposes of illustration, it will be apparent to those skilled in the art that the invention is susceptible to additional embodiments and that certain of the details herein may be varied considerably without departing from the basic principles of the invention.

1. A method comprising:
providing a first physiological fluid comprising cells of different sizes that include progenitor cells, fetal cells, stem cells or tumor cells to an array of traps positioned along multiple channels, the fluid provided at a pressure sufficient to cause vortex flow within the traps and trap larger cells including progenitor cells, fetal cells, stem cells or tumor cells present in the sample in the traps;
providing to at least some of the traps in the array a second fluid having a nucleic acid vector encoding a gene product or isolated protein for immortalization of the cells; and
applying a voltage across an electrode structure, the electrode structure to provide an electric field in the traps to cause electroporation of the trapped cells' membrane for intracellular delivery of the nucleic acid vector or isolated protein in the second fluid into the trapped cells.

2. The method of claim 1 wherein the physiological sample comprises blood, saliva, ascities fluid, cerebrospinal fluid or urine.

3. The method of claim 1 wherein the gene product or protein comprises TERT.

4. (canceled)

5. The method of claim 1 wherein the larger cells are circulating tumor cells, fetal cells, epithelial progenitor cells or endothelial progenitor cells.

6. The method of claim 1 wherein the electrode structure is formed with different connection widths.

7. The method of claim 1 wherein electrode dimensions are selected to promote variations in electric field profiles in different chambers to identify electrode dimensions that produce a desired electric field profile.

8. The method of claim 1 wherein the electrode structure has interdigitated electrodes formed in the traps.

9. A method comprising:
delivering a fluid having a physiological sample comprising cells of different sizes that include progenitor cells, fetal cells, stem cells or tumor cells to multiple traps via a channel connecting the traps;
maintaining a vortex flow in the traps to retain circulating progenitor cells, fetal cells, stem cells or tumor cells in the sample in the traps;
providing a fluid having a nucleic acid vector encoding a gene product, the expression of which in the cells immortalizes the cells, or isolated protein that immortalizes or provides for extended in vitro culturing of the cells, to the traps; and
providing an electric field across the traps to perform electroporation of the vector or the isolated protein into the cells in the traps.

10. The method of claim 9 wherein delivering the sample comprising cells is performed by transporting a first fluid solution containing the cells via the channel at a speed such that the fluid has a Reynolds number of greater than 100 to create the vortex flow in the traps.

11. The method of claim 9 wherein providing the vector or isolated protein to the traps comprises using a second fluid solution containing the vector or the isolated protein while maintaining the vortex flow in the traps and removing the first solution.

12. The method of claim 9 wherein the electric field across the traps is substantially uniform.

13. The method of claim 9 further comprising using a third fluid solution containing further molecules of interest while maintaining the vortex flow in the traps, the further molecules of interest being provided following electroporation of the vector or the isolated protein.

14. The method of claim 13 further comprising providing an electric field across the traps to perform electroporation of the further molecules of interest into the cells in the traps, wherein the electric field is adapted to enhance delivery of the further molecules of interest to the cells.

15. (canceled)

16. The method of claim 14 wherein the vortex flow is maintained during the entire method.

17. The method of claim 9 wherein delivering cells in solution to multiple traps via the channel connecting the traps includes providing the first solution to the channel via an inertial focusing region to cause the cells to move close to the sides of the channel via fluidic forces.

18. The method of claim 17 wherein the channel breaks into multiple channels, each having opposing pairs of traps disposed along a length of the channels.

19. The method of claim 9 wherein the sample comprises blood, saliva, ascities fluid, cerebrospinal fluid or urine.

20. The method of claim 9 wherein the gene product or protein comprises TERT.

21-22. (canceled)

23. The method of claim 9 wherein the electroporation produces immortalized cells from circulating tumor cells.

24. Isolated immortalized circulating tumor cells produced by the method of claim 23.

25-26. (canceled)
